(19) United States
(12) Patent Application Publication
(10) Pub. No.: US 2025/0361489 A1
(43) Pub. Date: Nov. 27, 2025

(54) METHOD FOR SIMULATING SENSITIVE RESPONSE OF JURKAT CELLS IN MECHANICAL MICROENVIRONMENTS

(71) Applicant: Changzhou University, Changzhou (CN)

(72) Inventors: Ming-Xing Ouyang, Changzhou (CN); Linhong Deng, Changzhou (CN); Hui Sheng, Changzhou (CN); Yaqin Li, Changzhou (CN)

(21) Appl. No.: 19/291,315

(22) Filed: Aug. 5, 2025

Related U.S. Application Data
(63) Continuation of application No. PCT/CN2023/119329, filed on Sep. 18, 2023.

(30) Foreign Application Priority Data
Mar. 14, 2023 (CN) 2023102403504

(51) Int. Cl. C12N 5/09; C12N 5/0783; C12N 15/87; G01N 21/64; G01N 33/68

(52) U.S. Cl. CPC C12N 5/0694; C12N 5/0636; C12N 15/87; G01N 21/64; G01N 33/68; C12N 2502/28; C12N 2533/54; G01N 2440/14; G01N 2474/10

(57) ABSTRACT

A method for simulating sensitive response of Jurkat cells in mechanical microenvironments, includes the following steps: S1, transfecting FRET probes into the Jurkat cells by using an electroporation method; S2, simulating the Jurkat cells in the mechanical microenvironments for a first time; S3, collecting FRET images of the Jurkat cells by using a FRET microscope; S4, performing quantitative analysis and statistical analysis on the FRET images; S5, simulating the Jurkat cells in the mechanical microenvironment for a second time; and S6, detecting an ERK phosphorylation level by using a Western blot method. By simulating different states of Jurkat cells, such as no-coated adhesion, charge adsorption, extracellular matrix adsorption, and endothelial cell layer adhesion, and by using methods of FRET living cell observation and biochemical detection method of ERK phosphorylation antibody, the method detects the Jurkat cells in different states, to obtain ERK activity of Jurkat cells in different mechanical microenvironments.

### CROSS-REFERENCE TO RELATED APPLICATIONS

This application is a continuation of International Patent Application No. PCT/CN2023/119329, filed on Sep. 18, 2023, which claims the priority of Chinese Patent Application No. 202310240350.4, filed on Mar. 14, 2023, both of which are herein incorporated by reference in their entirety.

### TECHNICAL FIELD

The disclosure relates to the field of biotechnologies, and more particularly to a method for simulating sensitive response of Jurkat cells in mechanical microenvironments.

### BACKGROUND

T cells are one of main components of a human immune system. In maintaining healthy immune responses, many T cells undergo significant changes in mechanical microenvironments, such as transitioning from a fluid state in a circulatory system to adhering to vessel walls, extravasating through blood vessels, and migrating within tissues to reach effector target sites. Jurkat cells belong to an acute T-cell leukemia cell line. As state and activity of the Jurkat cells in human bodies cannot be observed, the disclosure explores effect of mechanical environment changes on the Jurkat cell activity by simulating.

### SUMMARY

The disclosure aims to solve at least one of technical problems in the art.

Therefore, the disclosure provides a method for simulating sensitive response of Jurkat cells in mechanical microenvironments. The method for simulating the sensitive response of the Jurkat cells in the mechanical microenvironments has an advantage of simulating states of the Jurkat cells in human bodies.

The method for simulating the sensitive response of the Jurkat cells in the mechanical microenvironments provided by an embodiment of the disclosure includes the following steps: S1, transfecting extracellular signal-regulated protein kinase (ERK) fluorescent resonance energy transfer (FRET) probe into the Jurkat cells by using an electroporation method; S2, simulating the Jurkat cells in the mechanical microenvironments for a first time; S3, collecting FRET images of the Jurkat cells by using a FRET microscope; S4, performing quantitative analysis and statistical analysis on the FRET images; S5, simulating the Jurkat cells in the mechanical microenvironment for a second time; and S6, detecting an ERK phosphorylation level of the Jurkat cells by using a Western blot method.

In an embodiment, in the step S2, states of simulating the Jurkat cells in the mechanical microenvironment mainly simulate activity of the Jurkat cells in an adherent state. States of the Jurkat cells in the mechanical microenvironments (i.e., adherent state) mainly include a no-coated adhesion state, a charge adsorption state, an extracellular matrix adsorption state, a state of Jurkat cells embedded in type I collagens with different stiffness, and a state of Jurkat cells embedded in Matrigel® gels with different stiffness.

In an embodiment, the step S3 includes: sucking 100 microliters (μL) of Jurkat cell suspension to place on a glass-bottom area in a middle of a confocal dish with a diameter of 1.5 centimeters (cm); placing the confocal dish in a cell incubator mounted on the FRET microscope at 37 degrees Celsius (° C.) and 5% carbon dioxide (CO₂); selecting multiple fluorescent cell observation positions by an imaging software within 10 minutes (min); and collecting FRET images of the multiple fluorescent cell observation positions under the FRET microscope by using a 100-fold oil-immersion objective.

In an embodiment, step S5 includes steps S51 through S53. S51 is performing pretreatment, including: adding Poly-L-lysine with a concentration of 2 milligrams per milliliter (mg/mL) into a six-well plate for plate pretreatment, and placing the six-well plate in the cell incubator for incubating at 37° C. for 4 hours (h) to obtain a pretreated six-well plate; and calculating a required cell amount of the Jurkat cells according to an inoculation of 2×10⁵ cells for each well of the six-well plate; putting a Jurkat cell suspension containing the required cell amount of the Jurkat cells on a centrifuge to centrifuge at 1000 revolutions per minute (rpm) for 3 min to obtain a first supernatant and a first precipitation, removing the first supernatant; and adding a culture medium to the first precipitation to resuspend the Jurkat cells to obtain a pretreated Jurkat cell suspension. S52 is preparing suspended cell samples, including: adding 4 milliliters (mL) of the culture medium and 200 μL of the pretreated Jurkat cell suspension into each well of a non-treated six-well plate to obtain a first mixture; gently blowing the first mixture added in the non-treated six-well plate with the pipette to ensure that the Jurkat cells are evenly dispersed in the culture medium; placing the non-treated six-well plate added with the first mixture in the cell incubator to perform suspension culture of the Jurkat cells at 37° C. and 5% CO₂ for 30 min to obtain a cultured first mixture; centrifugating the cultured first mixture to obtain a second supernatant and a second precipitation, and removing the second supernatant; adding 100 μL of 2× sodium dodecyl sulfate-polyacrylamide gel electrophoresis (SDS-PAGE) sample loading buffer to the second precipitation to obtain a second mixture; fully lysing the second mixture on ice for 30 min to obtain a lysed second mixture; denaturing the lysed second mixture at 100° C. for 10 min to obtain a denatured second mixture; and centrifugating the denatured second mixture for 5 min to obtain a third supernatant as the suspended cell samples. S53 is preparing adherent cell samples, including: removing the Poly-L-lysine hydrobromide from the pretreated six-well plate to obtain a six-well plate with the Poly-L-lysine removed, adding 200 μL of the pretreated Jurkat cell suspension to each well of the six-well plate with the Poly-L-lysine removed for cell adherent culture to obtain a third mixture; placing a six-well plate with the third mixture in the cell incubator for incubating the Jurkat cells at 37° C. and 5% CO₂ for 30 min to obtain a cultured third mixture; taking a six-well plate with the cultured third mixture out of the cell incubator and gently blowing the cultured third mixture in the six-well plate with the pipette to make adherent cells slide off; collecting a cell suspension of the adherent cells, and centrifugating the cell suspension of the adherent cells to obtain a fourth supernatant and a third precipitation, and removing the fourth supernatant; adding 100 μL of 2×SDS-PAGE sample loading buffer to the third precipitation to obtain a fourth mixture; fully lysing the fourth mixture on ice for 30 min to obtain a lysed fourth mixture, denaturing the lysed fourth mixture at 100° C. for 10 min to obtain a denatured fourth mixture, and centrifugating the denatured fourth mixture at high speed for 5 min to obtain a fifth supernatant as the adherent cell samples.

In an embodiment, a forming process of the no-coated adhesion state specifically includes: sucking a certain amount of the Jurkat cell suspension to place on a glass-bottom area in a middle of a first confocal dish, and placing the first confocal dish in the cell incubator mounted on the FRET microscope. A forming process of the charge adsorption state specifically includes: adding a certain amount of Poly-L-lysine solution to a glass-bottom area of a second confocal dish to pretreat the second confocal dish in the incubator at 37° C. for 4 h; removing the Poly-L-lysine solution from the second confocal dish; and adding 200 μL of the Jurkat cell suspension to the second confocal dish. A surface of the Poly-L-lysine solution is positively charged, and surfaces of the Jurkat cells are negatively charged; and according to a principle of positive and negative charge attraction, the Jurkat cells are adhered to the glass-bottom area of the second confocal dish. A forming process of the extracellular matrix adsorption state specifically includes: adding fibronectin (FN, 20 μg/mL), type I collagen (100 μg/mL), or a Matrigel® solution (100 μg/mL) to a glass-bottom area of a third confocal dish for pretreatment to obtain a pretreated third confocal dish, and adding a small amount of the Jurkat cell suspension to the pretreated third confocal dish to promote the Jurkat cells to adhere to a matrix coating at the glass-bottom area of the pretreated third confocal dish. A forming process of an endothelial cell layer adhesion state specifically includes: inoculating human umbilical vein endothelial cells (HUVEC) onto a glass-bottom area of a fourth confocal dish, and adding a small amount of the Jurkat cell suspension into the fourth confocal dish when a cell density of the HUVEC reaches above 80%, to thereby achieve an effect of the Jurkat cells adhering to endothelial cells.

In an embodiment, a forming process of the state of the Jurkat cells embedded in type I collagens with different stiffness specifically includes: mixing 45 μL of type I collagen (COL) with different dilution concentrations with 5 μL of neutralization solution in Eppendorf (EP) tubes on ice, respectively; adding the Jurkat cells to the EP tubes and mixing uniformly to thereby obtain cell mixtures, wherein final concentrations of the COL are 2 mg/mL and 4 mg/mL respectively; then transferring, by the pipette, the cell mixtures to fifth confocal dishes; placing the fifth confocal dishes in the cell incubator at 37° C. for 15 min to form matrix gels; and adding 1 mL of the culture medium to the fifth confocal dishes with the matrix gels for microscopic imaging. A forming process of the state of the Jurkat cells embedded in Matrigel® gels with different stiffness specifically includes: aliquoting 100 μl of Matrigel® solutions with Matrigel® concentrations of 100% and 50% on ice, in which the Matrigel® solution with the Matrigel® concentration of 50% is diluted by phosphate buffered saline; and after centrifugating the Jurkat cell suspension in an EP tube at 1000 rpm for 1 min to obtain a cell pellet, resuspending the cell pellet with the Matrigel® solutions to obtain cell mixtures; transferring the cell mixtures to sixth confocal dishes, and placing the sixth confocal dishes in the cell incubator at 37° C. for 15 min; and adding 1 mL of the culture medium to the sixth confocal dishes for microscopic FRET imaging after the Matrigel® gels form.

The disclosure has the following beneficial effects. By simulating different states of the Jurkat cells, such as the no-coated adhesion state, the charge adsorption state, the extracellular matrix adsorption state, and the endothelial cell layer adhesion state, and by using a FRET living cell observation method and a biochemical detection method of ERK phosphorylation antibody along with total ERK immunostaining with ERK antibody as the input controls, the disclosure detects the Jurkat cells in different states, to thereby obtain the activity of Jurkat cells in different mechanical microenvironments.

Other features and advantages of the disclosure will be set forth in the following specification, and part of the features and advantages of the disclosure will be apparent from the specification or may be learned by embodiments of the disclosure. Objectives and other advantages of the disclosure are realized and obtained by structures specified in the specification, claims, and attached drawings.

To make the above objectives, features and advantages of the disclosure more apparent and easier to understand, the disclosure will be described in detail as follows with reference to embodiments and attached drawings.

### BRIEF DESCRIPTION OF DRAWINGS

Above or attached features and advantages of the disclosure will be apparent and easy to understand with reference to the following attached drawings.

FIG. 1 illustrates a schematic diagram of simulating Jurkat cells maintained in 10% fetal bovine serum (FBS) culture medium in an adherent state at different time points observed by a 40-fold microscope according to an embodiment of the disclosure.

FIG. 2 illustrates a schematic diagram of simulating the Jurkat cells maintained in 1% FBS culture medium in the adherent state at different time points observed by a 100-fold microscope according to the embodiment of the disclosure.

FIG. 3 illustrates a first data plot of ERK activity changing over time of simulating the Jurkat cells in the adherent state according to the embodiment of the disclosure.

FIG. 4 illustrates a second data plot of the ERK activity changing over time of simulating the Jurkat cells in the adherent state according to the embodiment of the disclosure.

FIG. 5 illustrates a schematic diagram of the Jurkat cells transfected with ERK FRET probes being inoculated onto a monolayer culture of human umbilical vein endothelial cells at different time points observed by the 100-fold microscope according to the embodiment of the disclosure.

FIG. 6 illustrates a data plot of ERK activity changing over time corresponding to FIG. 5.

FIG. 7 illustrates a data plot of ERK activity detected in 293T cells in an adherent state at different time points when the 293T cells are transfected with the ERK FRET probes and pre-cultured for 1 h and 6 h in suspension above 1% non-adhesive agarose gel.

FIG. 8 illustrates a data plot of Fyn kinase activity of the Jurkat cells transfected with Fyn FRET probes in an adherent state at different time points.

FIG. 9 illustrates first protein band patterns of phosphor-ERK (p-ERK) and total ERK amount (ERK) of the Jurkat cells in a suspension state and the adherent state, in which “Dmso” refers to addition of 0.1% dimethyl sulfoxide (DMSO) solvent in a culture medium.

FIG. 10 illustrates a data plot of ERK phosphorylation level of the Jurkat cells in comparison to the total ERK amount in the suspension state and the adherent state with suspension culture control normalized to 1.0, corresponding to FIG. 9.

FIG. 11 illustrates a second protein band patterns of the phospho-ERK and the total ERK amount of the Jurkat cells in the suspension state and the adherent state, in which “Agarose” refers to the Jurkat cells cultured on 1% non-adhesive agarose gel.

FIG. 12 illustrates a data plot of ERK phosphorylation level of the Jurkat cells in comparison to the total ERK amount in the suspension state and the adherent state with the suspension culture control normalized to 1.0, corresponding to FIG. 11.

FIG. 13 illustrates a first protein band patterns of the phospho-ERK and the total ERK amount of the Jurkat cells in the suspension state and the adherent state and inhibiting intracellular calcium channels (10 μM of inhibitor), in which “Nifedipine” refers to an L-type calcium channel inhibitor; 2-Aminoethyl diphenylborinate (2-APB) refers to an inositol triphosphate receptor (IP₃R) channel inhibitor; and “Thapsigargin” refers to a SarcoEndoplasmic Reticulum Calcium ATPase (SERCA) calcium pump inhibitor.

FIG. 14 illustrates a data plot of ERK phosphorylation level of the Jurkat cells in comparison to the total ERK amount in the suspension state and the adherent state with the suspension culture control normalized to 1.0, corresponding to FIG. 13.

FIG. 15 illustrates a second protein band patterns of the phospho-ERK and the total ERK amount of the Jurkat cells in the suspension state and the adherent state and activating cellular integrins with 0.5 millimoles per liter (mmol/L) manganese chloride (MnCl₂).

FIG. 16 illustrates a data plot of ERK phosphorylation level of the Jurkat cells in comparison to the total ERK amount in the suspension state and the adherent state with the suspension culture control normalized to 1.0, corresponding to FIG. 15.

FIG. 17 illustrates a second protein band patterns of the phospho-ERK and the total ERK amount of the Jurkat cells in the suspension state and the adherent state (blank, fibronectin, collagen).

FIG. 18 illustrates a data plot of ERK activity in the Jurkat cells at different time points when the Jurkat cells are embedded in type I collagen with COL concentrations of 2 mg/ml and 4 mg/mL.

FIG. 19 illustrates a data plot of ERK activity in the Jurkat cells at different time points when the Jurkat cells are embedded in Matrigel® gels with Matrigel® concentrations of 100% and 50%.

FIG. 20 illustrates a flowchart of the disclosure.

### DETAILED DESCRIPTION OF EMBODIMENTS

Embodiments of the disclosure will be described in detail as follows. Examples of the embodiments are illustrated in attached drawings. Throughout the attached drawings, same or similar reference numerals refer to same or similar elements or components having the same or similar functions. The embodiments described below with reference to the attached drawings are merely illustrative and are not intended to limit a scope of protection of the disclosure.

In description of the disclosure, it should be understood that, orientational or positional relationships indicated by terms “center”, “longitudinal”, “transverse”, “length”, “width”, “thickness”, “up”, “down”, “front”, “back”, “left”, “right”, “vertical”, “horizontal”, “top”, “bottom”, “inside”, “outside”, “clockwise”, “counterclockwise”, “axial”, “radial”, and “circumferential” are orientational or positional relationships illustrated in the attached drawings. These terms are only for convenience of describing the disclosure and simplifying the description and do not indicate or imply that referred devices or elements must have specific orientations or be constructed and operated in specific orientations, so they cannot be understood as limiting the disclosure. In addition, features defined as “first” and “second” may explicitly or implicitly include one or more of these features. In the description of the disclosure, unless otherwise indicated, “multiple” means two or more.

In the description of the disclosure, it should be understood that, unless otherwise specified and limited, terms “install”, “connect” and “link” should be understood in a broad sense. For example, it can be fixed connection, detachable connection or integrated connection; mechanical connection or electrical connection; direct connection, indirect connection through an intermediate medium, or internal connection between two components. For those skilled in the art, specific meaning of the above terms in the disclosure can be understood based on specific situations.

A method for simulating sensitive response of Jurkat cells in mechanical microenvironments provided by an embodiment of the disclosure will be described in detail as follows with reference to the attached drawings.

As illustrated in FIG. 19, the method for simulating the sensitive response of the Jurkat cells in the mechanical microenvironments provided by the embodiment of the disclosure includes the following steps S1 through S6. S1, ERK FRET probes are transfected into the Jurkat cells by using an electroporation method. S2, the Jurkat cells in the mechanical microenvironments is simulated for a first time. S3, FRET images of the Jurkat cells are collected by using a FRET microscope. S4, quantitative analysis and statistical analysis are performed on the FRET images. S5, the Jurkat cells in the mechanical microenvironments is simulated for a second time. S6, an ERK phosphorylation level of the Jurkat cells along with total ERK immunostaining is detected by using a Western blot method.

In the embodiment, the step S1 specifically includes steps S11 through S15. S11, 500 μL of Roswell Park Memorial Institute 1640 (RPMI-1640) culture medium with 10% FBS is added into each well of a 24-well cell culture plate by a pipette; and the 24-well cell culture plate is placed in a cell incubator for preheating to obtain a preheated culture medium. S12, a Jurkat cell suspension is taken by counting and according to an inoculation of 2×10⁵ Jurkat cells for each well of the 24-well cell culture plate; the Jurkat cell suspension is centrifuged to obtain the Jurkat cells pellet; and the Jurkat cells are washed with a phosphate buffered saline solution for twice to obtain washed Jurkat cells. S13, the washed Jurkat cells are resuspended with an electroporation buffer to obtain a resuspended Jurkat cell suspension; ERK FRET plasmid is added to the resuspended Jurkat cell suspension and mixed well to obtain a Jurkat cell suspension added with the ERK FRET plasmid; and 10 μL of the Jurkat cell suspension added with the ERK FRET plasmid is sucked by an electroporation pipette. S14, the electroporation pipette is inserted into an electroporation device for electroporation to thereby obtain a Jurkat cell suspension treated with electroporation, in which a voltage of the electroporation device is set to 1325 volts (V), a duration is set to 10 milli seconds (ms), and a pulse number is set to 3. S15, the Jurkat cell suspension treated with electroporation is transferred into the preheated culture medium, and the Jurkat cells are incubated at 37° C.

Further, in the step S12, a rotating speed of centrifugating is 1000 rpm and a duration of the centrifugating is 3 min. In step S13, a requirement is no bubble generation.

In the embodiment, in the step S2, states of simulating the Jurkat cells in the mechanical microenvironment mainly include: a no-coated adhesion state, a charge adsorption state, an extracellular matrix adsorption state, a state of Jurkat cells embedded in type I collagens with different stiffness, and a state of Jurkat cells embedded in Matrigel gels with different stiffness.

In the embodiment, a forming process of the no-coated adhesion state specifically includes the following steps. 100 μL of the Jurkat cell suspension is sucked to place on a glass-bottom area in a middle of a first confocal dish with a diameter of 15 millimeters (mm). The first confocal dish is placed in a cell incubator mounted on the FRET microscope. In the forming process, choosing a small amount of culture medium is helpful to reduce floating of Jurkat cells.

A forming process of the charge adsorption state specifically includes the following steps. 1 mL of a Poly-L-lysine solution with a concentration of 2 mg/mL is added to a glass-bottom area of a second confocal dish to pretreat the second confocal dish in the cell incubator at 37° C. for 4 h. The Poly-L-lysine solution is removed from the second confocal dish. 200 μL of the Jurkat cell suspension is added to the second confocal dish. A surface of the Poly-L-lysine solution is positively charged, and surfaces of the Jurkat cells are negatively charged; and according to a principle of positive and negative charge attraction, the Jurkat cells are adhered to the glass-bottom area of the second confocal dish.

A forming process of the extracellular matrix adsorption state specifically includes the following steps. Fibronectin (FN), type I collagen, or a Matrigel® solution is added to a glass-bottom area of a third confocal dish for pretreatment to obtain a pretreated third confocal dish. A small amount of the Jurkat cell suspension is added to the pretreated third confocal dish to promote the Jurkat cells to adhere to a matrix coating at the glass-bottom area of the pretreated third confocal dish. A forming process of an endothelial cell layer adhesion state specifically includes the following steps. HUVECs are inoculated onto a glass-bottom area of a fourth confocal dish. A small amount of the Jurkat cell suspension is added into the fourth confocal dish when a cell density of the HUVEC reaches above 80%, to thereby achieve an effect of the Jurkat cells adhering to endothelial cells.

A forming process of the state of the Jurkat cells embedded in type I collagens with different stiffness specifically includes the following steps. 45 μL of COL with different dilution concentrations are mixed with 5 μL of neutralization solution in EP tubes on ice, respectively. The Jurkat cells are added to the EP tubes and mixed uniformly to thereby obtain cell mixtures, in which final concentrations of the COL are 2 mg/mL and 4 mg/mL, respectively. The cell mixtures are transferred to fifth confocal dishes by the pipette. The fifth confocal dishes are placed in the cell incubator at 37° C. for 15 min to form matrix gels. 1 mL of the culture medium is added to the fifth confocal dishes with the matrix gels for microscopic imaging.

A forming process of the state of the Jurkat cells embedded in Matrigel® gels with different stiffness specifically includes the following steps. 100 μl of Matrigel® solutions with Matrigel® concentrations of 100% and 50% are aliquoted on ice. in which the Matrigel® solution with the Matrigel® concentration of 50% is diluted by phosphate buffered saline. After centrifugating the Jurkat cell suspension in an EP tube at 1000 rpm for 1 min to obtain a cell pellet, the cell pellet is resuspended with the Matrigel® solutions to obtain cell mixtures. The cell mixtures are transferred to sixth confocal dishes. The sixth confocal dishes are placed in the cell incubator at 37° C. for 15 min, in which the Matrigel® concentrations are 100% and 50%, respectively. 1 mL of the culture medium is added to the sixth confocal dishes for microscopic FRET imaging after the Matrigel® gels form.

In step S3, the FRET microscope is set to select two imaging channels, and the two imaging channels include an enhanced cyan fluorescent protein (ECFP) imaging channel and a FRET imaging channel. Fluorescence filter parameters for the ECFP imaging channel are: excitation at (436±10) nm (i.e., 426 nm to 446 nm), splitting at 455 nm, and emission at (480±20) nm (i.e., 460 nm to 500 nm). Fluorescence filter parameters for the FRET imaging channel are: excitation at (436±10) nm (i.e., 426 nm to 446 nm), splitting at 455 nm, and emission at (535±15) nm (i.e., 520 nm to 550 nm). During FRET imaging, the ECFP imaging channel and the FRET imaging channel are automatically and quickly switched by an imaging software to realize real-time acquisition of images of the ECFP imaging channel and the FRET imaging channel.

In the step S3, the Jurkat cells are imaged by FRET microscopy. 100 μL of the Jurkat cell suspension is sucked to place on a glass-bottom area in a middle of a seventh confocal dish; then the seventh confocal dish with the Jurkat cell suspension is placed in the cell incubator mounted on the FRET microscope at 37° C. and 5% CO₂. Multiple fluorescent cell observation positions are selected by the imaging software within 10 min. FRET images of the multiple fluorescent cell observation positions are collected under the FRET microscope by using a 40-fold or 100-fold oil-immersion objective.

In an embodiment, in step S4, the FRET images of the multiple fluorescent cell observation positions are analyzed by a FRET image analysis software Fluocell6.0.0 developed on a MATLAB software platform to obtain experimental analysis data. The experimental analysis data is expressed as mean+standard deviation and analyzed by statistical analysis software Origin8.0 and GraphPadPrism6.0. T-test is used to compare differences between data of each group, and a P value of the t-test is less than 0.05 for significant difference.

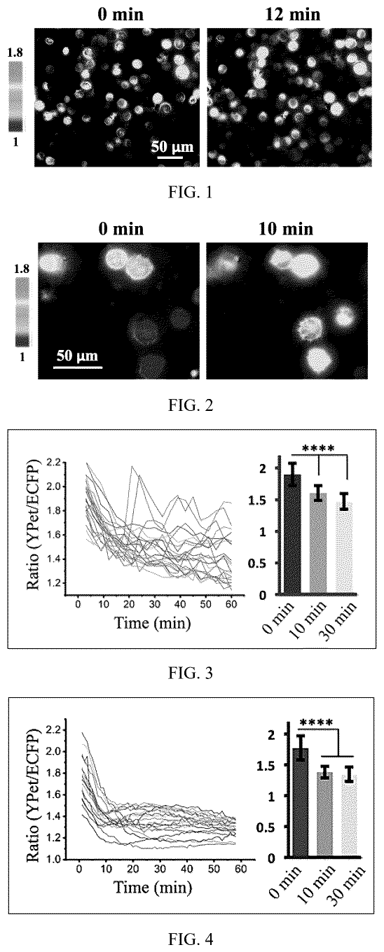

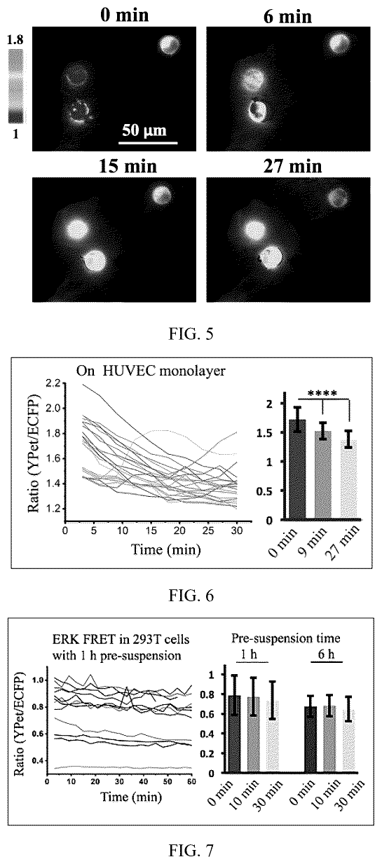

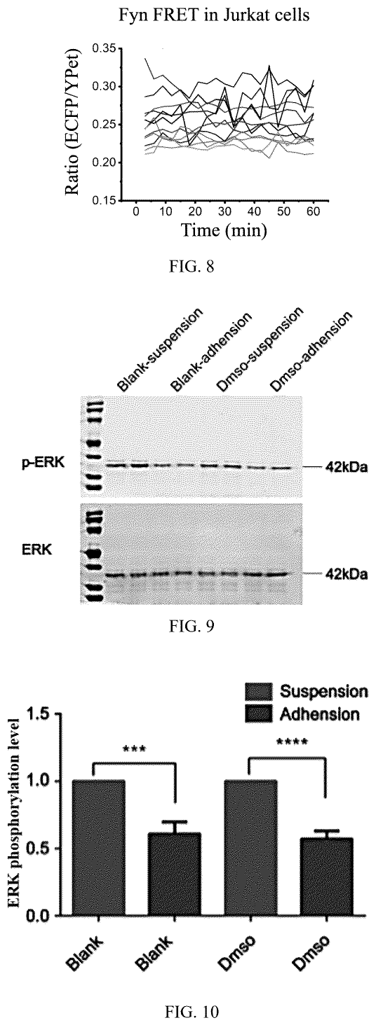

As illustrated in FIG. 1 through FIG. 4, activity of the Jurkat cells simulated in an adherent state decreases gradually over time. As illustrated in FIG. 5 and FIG. 6, ERK activity of the Jurkat cells transfected with ERK FRET probes and being inoculated onto monolayer culture of human umbilical vein endothelial cells still decreases over time. As illustrated in FIG. 7, ERK activity in 293T cells does not decrease upon contact with a hard matrix when the 293T cells are transfected with the ERK FRET probe and pre-cultured for 1 h or 6 h in suspension above 1% non-adhesive agarose gel. As illustrated in FIG. 8, when the Jurkat cells are transfected with other probes, such as Fyn FRET probe, cellular Fyn kinase is not as sensitive as ERK kinase and does not show downregulated activity upon contact with a hard matrix.

In an embodiment, step S5 includes steps S51 through S53. S51 is pretreating, including the following steps. Poly-L-lysine with a concentration of 2 mg/mL is added into a six-well plate for plate pretreatment. The six-well plate is placed in the cell incubator for incubating at 37° C. for 4 h to obtain a pretreated six-well plate. A required cell amount of the Jurkat cells is calculated according to an inoculation of 2×10⁵ cells for each well of the six-well plate. A Jurkat cell suspension containing the required cell amount of the Jurkat cells is put on a centrifuge to centrifuge at 1000 rpm for 3 min to obtain a first supernatant and a first precipitation. The first supernatant is removed. The culture medium is added to the first precipitation to resuspend the Jurkat cells to obtain a pretreated Jurkat cell suspension. S52 is preparing suspended cell samples, including the following steps. 4 mL of the culture medium and 200 μL of the pretreated Jurkat cell suspension are added into each well of a non-treated six-well plate to obtain a first mixture. The first mixture added in the non-treated six-well plate is gently blown with the pipette to ensure that the Jurkat cells are evenly dispersed in the culture medium. The non-treated six-well plate added with the first mixture is placed in the cell incubator to perform suspension culture of the Jurkat cells at 37° C. and 5% CO₂ for 30 min to obtain a cultured first mixture. The cultured first mixture is centrifuged to obtain a second supernatant and a second precipitation. The second supernatant is removed. 100 μL of 2×SDS-PAGE sample loading buffer is added to the second precipitation to obtain a second mixture. The second mixture is fully lysed on ice for 30 min to obtain a lysed second mixture. The lysed second mixture is denatured at 100° C. for 10 min to obtain a denatured second mixture. The denatured second mixture is centrifuged at high speed for 5 min to obtain a third supernatant as the suspended cell samples. S53 is preparing adherent cell samples, including the following steps. The Poly-L-lysine is removed from the pretreated six-well plate to obtain a six-well plate with the Poly-L-lysine removed. 200 μL of the Jurkat cell suspension are added to each well of the six-well plate with the Poly-L-lysine removed for cell adherent culture to obtain a third mixture. A six-well plate with the third mixture is placed in the cell incubator for incubating the Jurkat cells at 37° C. and 5% CO₂ for 30 min to obtain a cultured third mixture. A six-well plate with the cultured third mixture is taken out of the cell incubator. The cultured third mixture in the six-well plate is gently blown with the pipette to make adherent cells to slide off. A cell suspension of the adherent cells is collected. The cell suspension of the adherent cells is centrifuged to obtain a fourth supernatant and a third precipitation. The fourth supernatant is removed. 100 μL of 2×SDS-PAGE sample loading buffer is added to the third precipitation to obtain a fourth mixture. The fourth mixture is fully lysed on ice for 30 minutes to obtain a lysed fourth mixture. The lysed fourth mixture is denatured at 100° C. for 10 min to obtain a denatured fourth mixture. The denatured fourth mixture is centrifuged at high speed for 5 min to obtain a fifth supernatant as the adherent cell samples.

In step S6, the ERK phosphorylation level is detected by using the Western blot method, specifically including sub-step (1) through sub-step (8).

### Sub-Step (1), Preparing Gel.

Firstly, a long glass plate and a short glass plate are cleaned and rinsed with distilled water. After rinsing, the long glass plate and the short glass plate are stood on an absorbent paper. After drying moisture on the long glass plate and the short glass plate, a bottom of the long glass plate and a bottom of the short glass plate are aligned, and the long glass plate and the short glass plate are installed into a rubber rack and clamped tightly. The distilled water is injected between the long glass plate and the short glass plate for leakage detection for 5 min to 10 min. In the embodiment, 1.5 mm, 10-well glass plates are used. A concentration of resolving gel is 12%, and a concentration of stacking gel is 4%. The distilled water between the long glass plate and the short glass plate is removed, and the moisture is dried with the absorbent paper. When preparing the resolving gel with the concentration of 12%, a resolving gel solution can be shaken well immediately after adding N,N,N′,N′-tetramethylethylenediamine (TEMED), and then the resolving gel is prepared and can be poured. When pouring the resolving gel, a 10 mL pipette can be used to suck 8 mL of the resolving gel and pour the resolving gel along the long glass plate and the short glass plate. When a gel surface rises to a middle line of a green band, pouring is completed. A layer of water is coated on the gel surface for liquid sealing. When there is a refractive line between the water and the gel surface, it indicates that the resolving gel has solidified. The layer of water can be poured out, and the moisture can be dried by the absorbent paper after the resolving gel has fully solidified for 5 min. When preparing the stacking gel with the concentration of 4%, a stacking gel solution can be shaken well immediately after adding the TEMED, then the stacking gel is prepared and can be poured. A remaining space between the long glass plate and the short glass plate is filled with the stacking gel, and then a comb is inserted into the stacking gel. After the stacking gel solidifies, a process of preparing gel is completed and a gel plate is obtained.

### Sub-Step (2): Running the Gel.

The gel plate prepared in the sub-step (1) is installed onto a vertical electrophoresis device. Sodium dodecyl sulfate (SDS) electrophoresis buffer is added to both an inner tank and an outer tank of an electrophoresis tank. Before sample loading, both sides of the comb are held vertically with both hands, and the comb is gently pulled out of the gel plate. Markers and protein samples are added into wells. The electrophoresis tank is connected to an electrophoresis apparatus. A voltage of the electrophoresis apparatus is set to 80 V and adjusted to 120 V after 30 min. When bromophenol blue migrates to 1 cm from a bottom of the electrophoresis tank, electrophoresis is stopped.

### Sub-Step (3): Transferring Membrane.

A polyvinylidene fluoride (PVDF) membrane is used for transferring membrane. A sandwich structure for transferring membrane is assembled in a membrane transfer buffer solution. With a black side facing downward, sponge, four layers of filter paper, gel, PVDF membrane, four layers of filter paper, and sponge are placed in turn to form the sandwich structure, and a whole process is guaranteed to be free of bubbles. Then the sandwich structure is placed into a membrane transfer tank to ensure that a positive electrode and a negative electrode are placed correctly. A membrane transfer solution is poured into the membrane transfer tank, and the transferring membrane is performed under ice bath conditions. Membrane transfer conditions include: 300 milliamperes (mA) of current and 90 min of duration.

### Sub-Step (4): Blocking.

After the transferring membrane is completed, the PVDF membrane is removed from the membrane transfer tank and immersed into a 5% bovine serum albumin (BSA) blocking solution in a box. The blocking solution is shaken slowly for 1 h. It should be noted that: a side of the PVDF membrane in contact with the gel is marked as a front side.

### Sub-Step (5): Primary Antibody Binding.

Firstly, the blocking solution is removed from the box, 5 mL (1:1000) of primary antibody diluent is added to the box until covering the PVDF membrane, and the box is left overnight at 4° C.

### Sub-Step (6): Secondary Antibody Binding.

Firstly, the primary antibody diluent is collected. The PVDF membrane is washed with 10 mL of 1×tris buffered saline with polysorbate 20 (i.e., TWEEN®-20) (TBST) for 5 min for 3 times. 5 mL (1:10000) fluorescent secondary antibody diluent is added to the PVDF membrane for incubating at the room temperature for 1 h in dark. The secondary antibody diluent is collected. 10 mL of 1×TBST is added to the secondary antibody diluent to wash the PVDF membrane for 5 min for 3 times.

### Sub-Step (7): Color Rendering.

The PVDF membrane is placed on an infrared laser imager for exposure imaging. It should be noted that: the front side of the PVDF membrane is placed facing downward.

### Sub-Step (8), Data Processing.

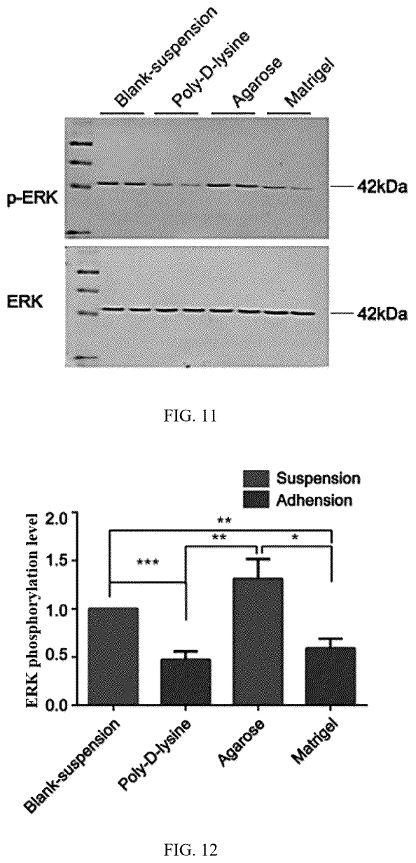

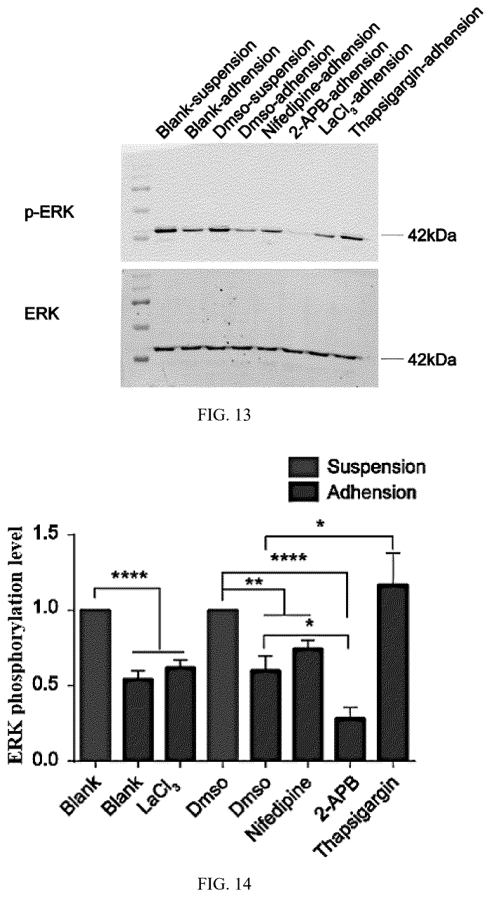

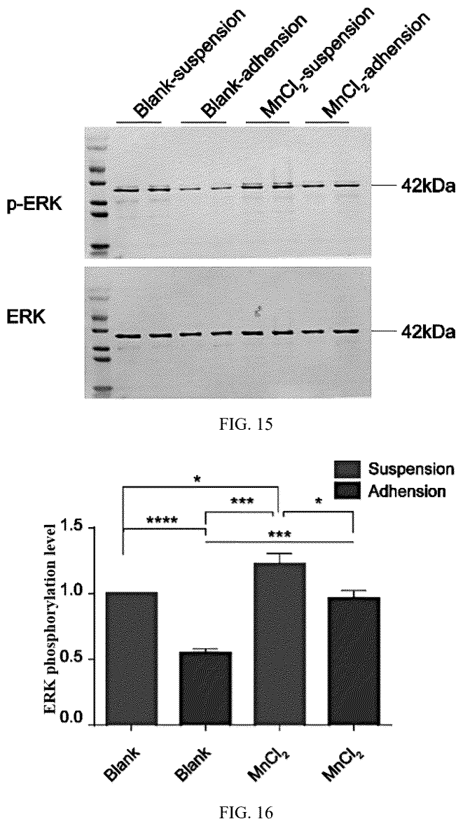

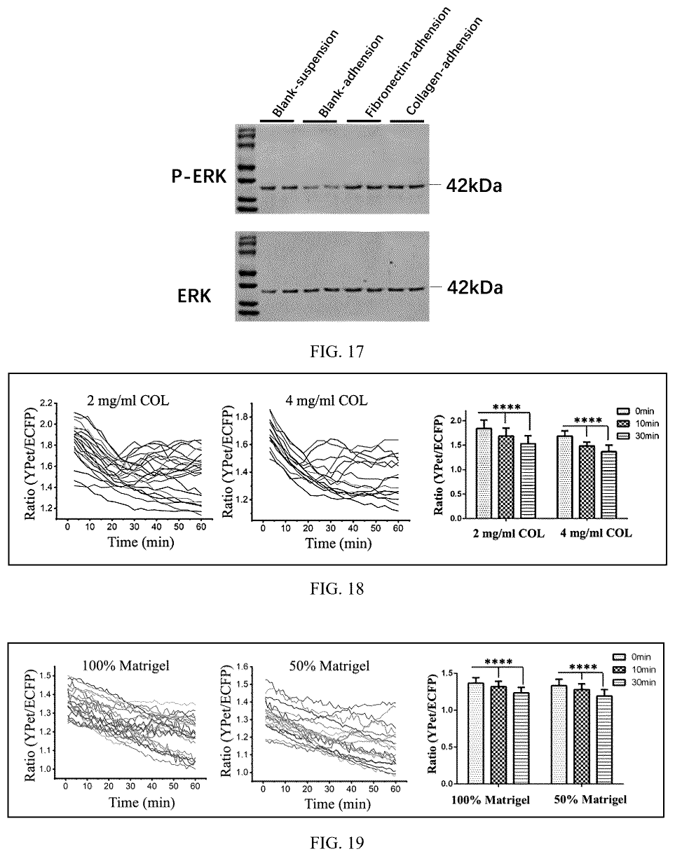

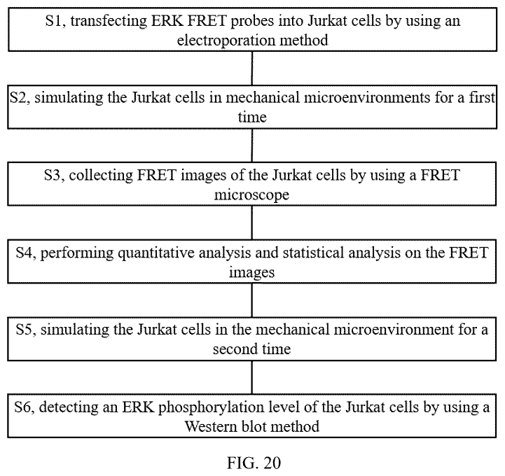

As illustrated in FIG. 9 through FIG. 12, when the ERK activity level of Jurkat cells is detected by ERK phosphorylation antibody, the ERK activity in the Jurkat cells decreases rapidly when the state of Jurkat cells changes from suspension culture to charged physical adsorption and adhesion on a glass surface. As illustrated in FIG. 13 through FIG. 16, after inhibiting calcium signals or activating integrins in the Jurkat cells, a decreasing trend of the ERK activity is reversed.

The disclosure has the following beneficial effects. By simulating different states of the Jurkat cells, such as no-coated adhesion, charge adsorption, extracellular matrix adsorption, and endothelial cell layer adhesion, and by using a FRET living cell observation method and a biochemical detection method of ERK phosphorylated antibody, the disclosure detects the Jurkat cells in different states, to thereby obtain the activity of Jurkat cells in different mechanical microenvironments. In this way, the disclosure provides supports for subsequent researches on T-cell leukemia, T-cell signaling transduction, and sensitivity of cancer cells to drug therapy.

In the description of this specification, description of reference terms “one embodiment”, “some embodiments”, “illustrative embodiments”, “examples”, “specific examples”, or “some examples” means that specific features, structures, materials, or characteristics described in conjunction with the embodiments or examples are included in at least one embodiment or example of the disclosure. In the specification, illustrative expressions of these above terms may not necessarily refer to the same embodiments or examples. Moreover, the specific features, structures, materials, or characteristics described can be combined in any one or more embodiments or examples in a suitable manner.

Although the embodiments of the disclosure have been illustrated and described, those skilled in the art should understand that many changes, modifications, replacements, and variations can be made to the embodiments without departing from principles and purposes of the disclosure, and the scope of protection of the disclosure is limited by the claims and their equivalents.

What is claimed is:

1. A method for simulating sensitive response of Jurkat cells in mechanical microenvironments, comprising the following steps:
S1, transfecting extracellular signal-regulated protein kinase (ERK) fluorescent resonance energy transfer (FRET) probes into the Jurkat cells by using an electroporation method;
S2, simulating the Jurkat cells in the mechanical microenvironments for a first time;
S3, collecting FRET images of the Jurkat cells by using a FRET microscope;
S4, performing quantitative analysis and statistical analysis on the FRET images;
S5, simulating the Jurkat cells in the mechanical microenvironment for a second time; and
S6, detecting an ERK phosphorylation level of the Jurkat cells by using a Western blot method.

2. The method for simulating the sensitive response of the Jurkat cells in the mechanical microenvironments as claimed in claim 1, wherein, in step S2, states of simulating the Jurkat cells in the mechanical microenvironments comprise: a no-coated adhesion state, a charge adsorption state, an extracellular matrix adsorption state, a state of Jurkat cells embedded in type I collagens with different stiffness.

3. The method for simulating the sensitive response of the Jurkat cells in the mechanical microenvironments as claimed in claim 1, wherein the step S3 comprises:
sucking 100 microliters (μL) of Jurkat cell suspension to place on a glass-bottom area in a middle of a confocal dish, and placing the confocal dish with the Jurkat cell suspension in a cell incubator mounted on the FRET microscope at 37 Celsius degrees (° C.) with 5% carbon dioxide (CO₂); and selecting a plurality of fluorescent cell observation positions by an imaging software within 10 minutes (min), and collecting the FRET images at the plurality of fluorescent cell observation positions under the FRET microscope by using a 100-fold oil-immersion objective.

4. The method for simulating the sensitive response of the Jurkat cells in the mechanical microenvironments as claimed in claim 1, wherein step S5 comprises the following steps:
S51, performing pretreatment, comprising: adding Poly-L-lysine with a concentration of 2 milligrams per milliliter (mg/mL) into a six-well plate for plate pretreatment, and placing the six-well plate in a cell incubator for incubating at 37° C. for 4 hours (h) for suspend cell adherent culture to obtain a pretreated six-well plate; and calculating a required cell amount of the Jurkat cells according to an inoculation of 2×10⁵ cells for each well of the six-well plate, putting a Jurkat cell suspension containing the required cell amount of the Jurkat cells on a centrifuge to centrifugate at 1000 revolutions per minute (rpm) for 3 min to obtain a first supernatant and a first precipitation, removing the first supernatant, and adding a culture medium to the first precipitation to resuspend the Jurkat cells to obtain a pretreated Jurkat cell suspension;
S52: preparing suspended cell samples, comprising: adding 4 milliliters (mL) of the culture medium and 200 μL of the pretreated Jurkat cell suspension into each well of a non-treated six-well plate to obtain a first mixture, and blowing the first mixture added in the non-treated six-well plate with a pipette to ensure that the Jurkat cells are evenly dispersed in the culture medium; placing the non-treated six-well plate added with the first mixture in the cell incubator to perform suspension culture of the Jurkat cells at 37° C. and 5% CO₂ for 30 min to obtain a cultured first mixture; centrifugating the cultured first mixture to obtain a second supernatant and a second precipitation, and removing the second supernatant; adding 100 μL of 2×sodium dodecyl sulfate-polyacrylamide gel electrophoresis (SDS-PAGE) sample loading buffer to the second precipitation to obtain a second mixture; fully lysing the second mixture on ice for 30 min to obtain a lysed second mixture; denaturing the lysed second mixture at 100° C. for 10 min to obtain a denatured second mixture; and centrifugating the denatured second mixture for 5 min to obtain a third supernatant as the suspended cell samples; and
S53, preparing adherent cell samples, comprising: removing the Poly-L-lysine from the pretreated six-well plate to obtain a six-well plate with the Poly-L-lysine removed, adding 200 μL of the pretreated Jurkat cell suspension to each well of the six-well plate with the Poly-L-lysine removed for cell adherent culture to obtain a third mixture, and placing a six-well plate with the third mixture in the cell incubator for incubating the Jurkat cells at 37° C. and 5% CO₂ for 30 min to obtain a cultured third mixture; taking a six-well plate with the cultured third mixture out of the cell incubator, and blowing the cultured third mixture in the six-well plate with the pipette to make adherent cells slide off; collecting a cell suspension of the adherent cells, centrifugating the cell suspension of the adherent cells to obtain a fourth supernatant and a third precipitation, and removing the fourth supernatant; adding 100 μL of 2×SDS-PAGE sample loading buffer to the third precipitation to obtain a fourth mixture; fully lysing the fourth mixture on ice for 30 min to obtain a lysed fourth mixture, denaturing the lysed fourth mixture at 100° C. for 10 min to obtain a denatured fourth mixture, and centrifugating the denatured fourth mixture for 5 min to obtain a fifth supernatant as the adherent cell samples.

5. The method for simulating the sensitive response of the Jurkat cells in the mechanical microenvironments as claimed in claim 2, wherein a forming process of the no-coated adhesion state specifically comprises: sucking a Jurkat cell suspension to place on a glass-bottom area in a middle of a first confocal dish with a diameter of 1.5 centimeters (cm), and placing the first confocal dish in a cell incubator mounted on the FRET microscope;
a forming process of the charge adsorption state specifically comprises: adding a Poly-L-lysine solution to a glass-bottom area of a second confocal dish to pretreat the second confocal dish in the cell incubator at 37° C. for 4 h, and removing the Poly-L-lysine solution from the second confocal dish; and adding 200 μL of the Jurkat cell suspension to the second confocal dish; wherein a surface of the Poly-L-lysine solution is positively charged, and surfaces of the Jurkat cells are negatively charged; and according to a principle of positive and negative charge attraction, the Jurkat cells are adhered to the glass-bottom area of the second confocal dish;
a forming process of the extracellular matrix adsorption state specifically comprises: adding fibronectin (20 μg/mL), type I collagen (100 μg/mL), or a Matrigel® (100 μg/mL) solution to a glass-bottom area of a third confocal dish for pretreatment to obtain a pretreated third confocal dish, and adding the Jurkat cell suspension to the pretreated third confocal dish to promote the Jurkat cells to adhere to a matrix gel at the glass-bottom area of the pretreated third confocal dish; and
a forming process of an endothelial cell layer adhesion state specifically comprises: inoculating human umbilical vein endothelial cells (HUVEC) onto a glass-bottom area of a fourth confocal dish, and adding the Jurkat cell suspension into the fourth confocal dish when a cell density of the HUVEC reaches above 80%, to thereby achieve an effect of the Jurkat cells adhering to endothelial cells.

6. The method for simulating the sensitive response of the Jurkat cells in the mechanical microenvironments as claimed in claim 2, wherein a forming process of the state of the Jurkat cells embedded in type I collagens with different stiffness specifically comprises: mixing 45 μL of collagens (COL) with different dilution concentrations with 5 μL of neutralization solution in Eppendorf (EP) tubes on ice, respectively; adding the Jurkat cells to the EP tubes and mixing uniformly to thereby obtain cell mixtures, wherein final concentrations of the COL are 2 mg/ml and 4 mg/mL respectively; transferring, by a pipette, the cell mixtures to fifth confocal dishes, placing the fifth confocal dishes in a cell incubator at 37° C. for 15 min to form matrix gels, and adding 1 mL of culture medium to the fifth confocal dishes with the matrix gels for microscopic imaging; and
a forming process of the state of the Jurkat cells embedded in Matrigel® gels with different stiffness specifically comprises: aliquoting 100 μl of Matrigel® solutions with Matrigel® concentrations of 100% and 50% on ice, in which the Matrigel® solution with the Matrigel® concentration of 50% is diluted by phosphate buffered saline; and after centrifugating the Jurkat cell suspension in an EP tube at 1000 rpm for 1 min to obtain a cell pellet, resuspending the cell pellet with the Matrigel® solutions to obtain cell mixtures; transferring the cell mixtures to sixth confocal dishes, and placing the sixth confocal dishes in the cell incubator at 37° C. for 15 min, wherein the Matrigel® concentrations are 100% and 50%, respectively; and adding 1 mL of the culture medium to the sixth confocal dishes for microscopic imaging after the Matrigel® gels form.
